(19) United States
(12) Patent Application Publication
(10) Pub. No.: US 2016/0235940 A1
(43) Pub. Date: Aug. 18, 2016

(54) DEVICE AND METHOD FOR SELECTIVE WAVELENGTH FILTRATION AND SELECTIVE WAVELENGTH TRANSMISSION FOR THERAPEUTIC EFFECT

(71) Applicant: Daniel Andrew Koifman, Brooklyn, NY (US)

(72) Inventor: Daniel Andrew Koifman, Brooklyn, NY (US)

(21) Appl. No.: 14/624,319

(22) Filed: Feb. 17, 2015

(51) Int. Cl. A61M 21/02; A61F 9/04

(52) U.S. Cl. CPC A61M 21/02; A61F 9/04; A61M 2021/0044

(57) ABSTRACT

The present invention generally relates to methods for providing darkness therapy for altering biological rhythms. Specifically, embodiments of the present invention are directed to the process of employing an optical device configured to block certain wavelengths of light while allowing other wavelengths to pass through in order to provide significant biologically therapeutic effects. These and other advantages are detailed further herein.

### FIELD OF THE INVENTION

The present invention generally relates to method for providing darkness therapy to alter biological rhythms and physiological processes. Specifically, this invention relates to a method for blocking and transmitting light of certain wavelengths for the purpose of providing a therapeutic effect.

### BACKGROUND OF THE INVENTION

Until the advent of the light bulb just over a hundred years ago, when humans have experienced light at night throughout our evolution in the color of night time firelight, namely, at least one or a combination of the following visible spectrums: yellow (570-590 nm), orange (590-620 nm), red (620-750 nm). Fire light may include infrared (700 nm-1 mm) light. However, since the invention of the light bulb, humans have been significantly exposed to the full spectrum of visible light at night, which includes spectrums in firelight as well as the ultraviolet, violet, blue and green spectrums (the spectrums contained in wavelengths of about 580 nanometers and below). As a consequence of such exposure to the full spectrum of light at night hours, human health has experienced detrimental effects from such full spectrum, artificial light.

Energy efficient lightbulbs (CFLs, LEDs) save on electricity but transmit dramatically different wavelengths that contain significantly more blue and green wavelength of light. Light pollution is having a dramatic increase. The National Park Service reported an image projection showing that by 2025 major urban areas of the United States will be 81-243 times the natural brightness level. The incandescent bulbs that more closely mimicked fire light than energy efficient lightbulbs have been banned from manufacture and sale in the US. The wavelengths in these new energy efficient bulbs have more potential to disrupt the biological rhythms and physiological processes of the body.

To illustrate the disruptive nature of green/blue light wavelengths of light, the World Health Organization has classified night-shift work as a probable human carcinogen, in part due to the ensuing circadian disruption brought upon by artificial light at night. Moreover, circadian disruption has been attributed to the increase of risk of heart disease, depression, learning issues, diabetes, obesity, metabolic syndrome and many other conditions currently plaguing modern societies.

Therefore, it is an object of the present invention to transmute natural and artificial light, including light at night and sunlight to transmit wavelengths and colors of light more commonly found in firelight to provide an effect approaching darkness or dim lighting to regulate biological rhythms and numerous physiological processes. It may be used at night to shield from common sources of light at night such as light bulbs, electronics, televisions and more. It may also be used during the day for purposes of adapting the body to different time zones and for shiftworkers and others that choose to sleep during the day and work and have more activity at night by blocking short and moderate wavelengths of light along with ultraviolet light. Embodiments of the present invention describe an apparatus and method to simulate “virtual darkness” to a recipient by transmitting spectrums of light commonly found in a long-wavelength of light, which significantly includes wavelengths found in natural fire light, while simultaneously blocking wavelengths of light that disrupt biological rhythms. These and other features and advantages of the present invention will be explained and will become obvious to one skilled in the art through the summary of the invention that follows.

### SUMMARY OF THE INVENTION

Accordingly, it is an object of the present invention to provide a method for providing darkness therapy for altering biological rhythms comprising of covering an individual's eyes with an optical device, configured to block at least 90% or more light at wavelengths in the range of 495-570 nm (+/−15 nm) and transmit light in at least one of orange (590-620 nm), red (620-750 nm), and near infrared light range spectrum (750 nm or greater), wherein said eyes are covered by said optical device for 2-4 hours before bed time to normalize said human being's circadian rhythm, wherein said optical devise comprises of red-colored lenses.

According to an embodiment of the present invention, the method is further configured to block at least 90% or more light at wavelengths in the range of 450 nm-495 nm.

According to an embodiment of the present invention, the method is further configured to block at least 90% or more light at wavelengths in the range of 380 nm-495 nm.

According to an embodiment of the present invention, covering the eyes with said device increases the level of at least one or more of melatonin and leptin.

According to an embodiment of the present invention, covering the eyes with said device decreases the level of one or more corticosteroid.

According to an embodiment of the present invention, covering the eyes with said device decreases the level of ghrelin.

According to an embodiment of the present invention, covering the eyes with said device assists with at least one of the following: weight management, sleep management, relaxation or stress management.

According to an embodiment of the present invention, covering the eyes with said device to treat at least one type of eating disorder.

According to an embodiment of the present invention, covering the eyes with said device for enriching melatonin in breast milk, wherein said optical devices are worn for at least two to four hours at night.

According to an embodiment of the present invention, covering the eyes with said optical device for use in treating jet leg, wherein the optical devise is worn an hour earlier every week or every day to prepare the body before flight. It may be worn an hour or more earlier before bedtime for a day or a period of days before timezone travel, to assist the body in alerting its clock and ameliorating jet lag. It may be worn before or during travel to help sync the biological clock to a destination where it is presently dark as the body clock is resistant to abrupt changes, this may help speed up the adaptation process upon arriving to the destination. It may be worn once arriving to a destination to assist with accommodating new desired local time or schedule by simulating darkness.

According to an embodiment of the present invention, covering the eyes with said optical device for use as an add-on treatment for a class of affective disorders, including bipolar disorder, anxiety, and depression.

According to an embodiment of the claimed invention, an optical device for providing darkness therapy for altering biological rhythms comprising a filter, configured to block at least 90% or more light at wavelengths in the range of 495-570 nm and transmit light in at least one of yellow (570-590 nm), orange (590-620 nm), red (620-750 nm), and near infrared light range spectrum (750 nm or greater), wherein said filter is a red-colored lens.

According to an embodiment of the claimed invention, an optical device for providing darkness therapy for altering biological rhythms further configured to block at least 90% or more light at wavelengths in the range of 450 nm-495 nm.

According to an embodiment of the claimed invention, an optical device for providing darkness therapy for altering biological rhythms further configured to block at least 90% or more light at wavelengths in the range of 380 nm-495 nm.

According to an embodiment of the claimed invention, an optical device for providing darkness therapy for altering biological rhythms further configured to block at least 90% or more light at wavelengths below 380 nm.

According to an embodiment of the claimed invention, the optical device is embodied in a pair of glasses.

### BRIEF DESCRIPTION OF THE DRAWINGS

FIG. 1 is a process flow of an exemplar method in accordance with embodiments of the present invention.

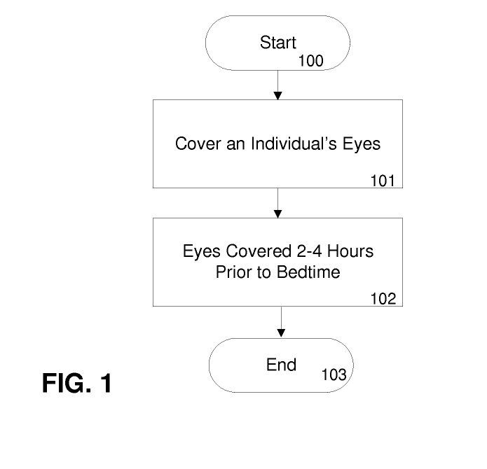

FIG. 2 is a front perspective view of an optical device for providing darkness therapy in accordance with an embodiment of the present invention.

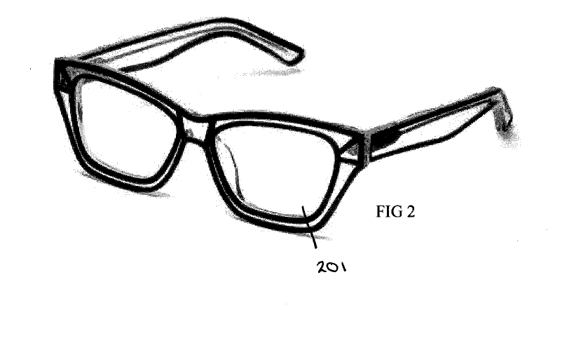

FIG. 3 is a process flow of an exemplary method in accordance with an embodiment of the present invention.

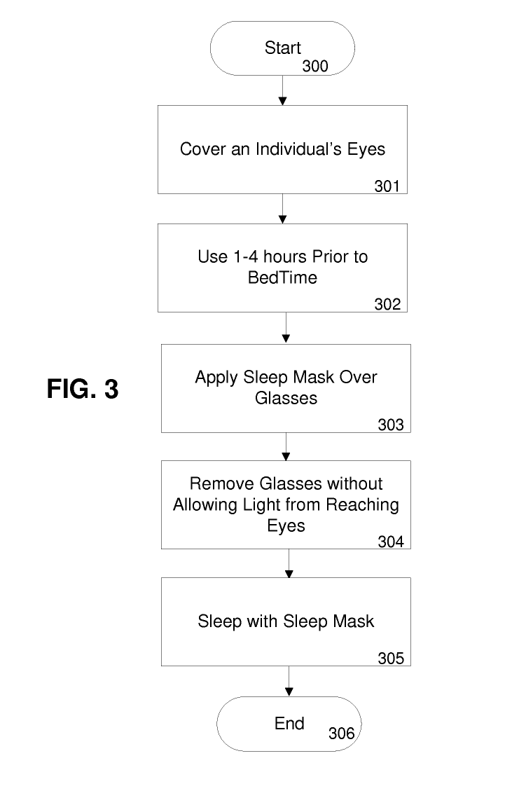

FIG. 4 is a process flow of an exemplary method in accordance with an embodiment of the present invention.

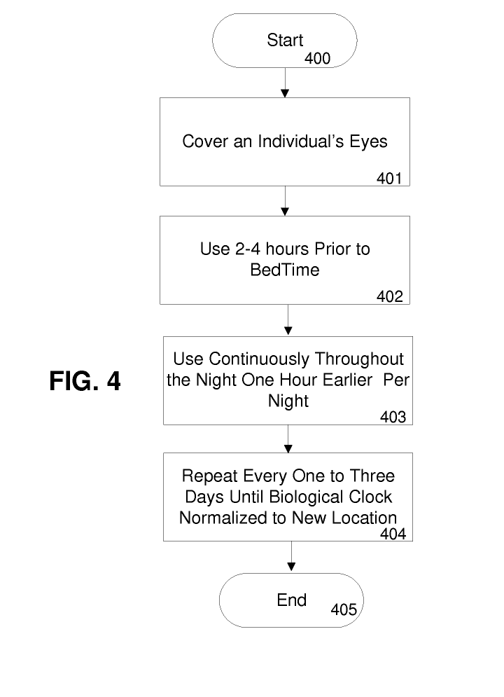

### DETAILED SPECIFICATION

The present invention relates to the field of optical devices. Specifically, embodiments of the present invention provide a method and apparatus for selectively blocking and selectively transmitting certain wavelengths of light in the visible and near infrared light spectrum.

According to an embodiment of the claimed invention, the apparatus and method is configured to block 90% or more of one or more ranges of wavelengths of light at or below green light while transmitting light at yellow light or above.

Sensitivity curves for photopic and scotopic photoreceptors reveal that green light and blue light are perceived by the human eye to be relatively brighter in both well-lit and low-light conditions. This suggests that the human eye receptors have evolved to perceive both green and blue light with greater sensitivity relative to other colors. Accordingly, the presence of such colors, in night conditions, can be disruptive to normal physiological rhythms.

### Exemplary Embodiment

According to an exemplary embodiment of the claimed invention, the method begins at step 100. At this step, the person may be experiencing some malady such as jet lag, inability to sleep, or inability to control appetite.

At step 101, the individual wears the optical device. In one embodiment, at step 102, the individual wears the device for 2-4 hours prior to bedtime. In other embodiments, the individual may wear the device for 2-4 hours during the day. At step 103, the method terminates.

According to an alternative embodiment of the claimed invention, the method can be used at sleep/night time with a sleep mask. This is a significant variation since light is can still permeate through closed eyes. In this embodiment, the method begins at step 300. An individual uses a glass embodiment of the apparatus in step 301 and covers his/her own eyes. At step 302, the glasses are worn 1-4 hours prior to bedtime or sleeptime. At step 303, a sleep mask is worn over the glasses. The glasses are removed from underneath the sleep mask at step 304 to minimize light accessing the individual's eyes. The individual is then instructed to sleep throughout the night at step 305. At step 306 the method terminates.

According to an alternative embodiment of the claimed invention, the method can be employed in situations where the individual is or is anticipating experiencing jet lag. In this embodiment, the method begins at step 400. At step 401, the individual covers his/her eyes and at step 402, the individual covers his eyes for 2-4 hours prior to bedtime. At step 403, the individual uses the apparatus continuously through the night. Each successive night, the method is followed one hour earlier. At step 404, this is repeated one to three days until the individual's biological clock normalizes to the new location upon travel. At step 405, the method terminates.

According to an alternate embodiment of the claimed invention, the method for jetlag can be used in tandem with the method as used with a sleep mask.

According to an embodiment of the present invention, the apparatus and method provide a therapeutic effect by providing an effect which comes close to approximating or replicating natural darkness in subjects but still granting them the ability to see and function through the invention by blocking unwanted circadian affecting spectrums from reaching photoreceptors in the eye. This invention comprises use of an optical device to filter light such that the light simulates an affect approaching darkness to facilitate the normalization of circadian and biological rhythms involved in numerous biological processes and health conditions.

According to an embodiment of the present invention, the apparatus and method yield therapeutic effects by regulating hormones. For example, the effect of modulating at least one of the following: melanopsin, melatonin, ghrelin, leptin and/or one or more glucocorticoid such as cortisol and may result in: promoting sleep, adjusting to jet lag, increasing endogenous antioxidants including melatonin in various parts of the body, assisting with curbing food cravings and binge eating, ameliorating some of the health hazards associated with light at night, and increasing the secretion of melatonin and/or decreasing one or more glucocorticoids such as cortisol in mother's milk which when ingested at night may help promote sleep and help set the inner clock of children consuming breast milk and may act as a neuroprotective agent, among other benefits.

According to an embodiment of the claimed inventions, the levels of human melatonin levels increase when blocking green light. When a person is exposed to green light, the production of melatonin is suppressed. By blocking artificial green light, the level of melatonin output is normalized.

The normalization of melatonin assists with setting biological clock which may include both the daily or biological circadian clock and circannual seasonal clock sleepiness and symptoms associated with such a condition. A relatively high output of melatonin is correlated with increase sleepiness. Conversely, individuals who are unable to sleep and are exposed to bright and/or short to moderate wavelength light have suppressed melatonin production. Accordingly, blocking any existing green light, which suppresses melatonin output will have the effect of increasing melatonin production, thereby facilitating healthy biological rhythms and this promoting healthy sleep management and sleepiness at night or other desired periods such as during the day for shiftworkers that sleep during the day and work at night.

The regulation of melatonin can also be applied for the treatment of epileptic seizures. As stated earlier, melatonin is an important hormone in the sleep-wake cycle. Detrimental effects of inadequate sleep include drowsiness, memory problems and epileptic seizures. Accordingly, the longer an individual goes without sleep, the increase likelihood that such individual may suffer an epileptic seizure. By normalizing melatonin levels for treating the sleep-wake cycle, the method is also able to decrease the incidents of epileptic seizure frequencies with no side-effects.

In addition to affecting sleepiness, normalization of melatonin levels can assist in providing therapeutic benefits for age-related neurodegenerative diseases. A physiological component of neurodegenerative diseases is the excessive production of free radicals. Antioxidants defend against the accumulation of free radicals by either preventing their formation or removing them before they become toxic to bodily systems. Studies have shown that an increase in melatonin has the effect of also increasing the levels of antioxidants. Accordingly, the method's increase in melatonin levels has the therapeutic effects of neutralizing or delaying any deleterious effects of free radicals and reactive oxygen species within the human body.

Similarly, the regulation of melatonin and its accompanying antioxidant properties through the claimed invention is also therapeutic to the intestinal organ. One of the disorders that can affect the intestine is the loss in ability to absorb nutritive calcium. If the calcium is not absorbed, it cannot be utilized for the numerous biological needs of the human body, including the formation and strengthening of bones, conducting nerve impulses, maintaining a normal heart beat. The loss of calcium absorption is attributed to the increase of oxidative stress and apoptosis upon the intestinal cells. Melatonin is able to trigger anti-apoptotic activity within cells. Accordingly, increasing the levels of melatonin counteracts the loss of calcium absorption and helps to restore regular intestinal functions.

According to an embodiment of the claimed invention, the apparatus and method decrease the level of at least one type of corticosteroid, such as cortisol. Cortisol is a steroid hormone produced by the adrenal gland and released upon triggering perceived stress from the hypothalamus. In night-time conditions without additional artificial light, cortisol levels have been shown to be at the lowest levels in normal healthy individuals. However, with the exposure of additional light, particularly green light at 555 nm promotes cortisol production which may not be healthful at night. Accordingly, by blocking out at least the green light at 555 nm (as well as light in the spectrum of green, blue, and violet light in the range of 570 nm and below), the levels of cortisol may remain relatively low or move downwards if the situation allows.

According to an embodiment of the claimed invention, the apparatus and method increase the hormone leptin and decreases the hormone ghrelin. Both hormones are associated with the modulation of hunger signaling and weight homeostasis. Significantly, hunger itself is a physiological mechanism that associatively cycles with an individual's sleep cycle. Sleep deprivation results in a reduction of leptin and an increase ghrelin. By normalizing an individual's sleep cycle through the claimed invention by blocking blue, green, and/or violet light and transmitting light in red, orange, and/or yellow spectrum, the individual's sleep and dietary cycle are simultaneous normalized.

Regulation of leptin may aid in the feeding cycle, as well as have effects in the circadian, wakefulness-sleep system. The energy homeostasis of an individual is maintained through the interaction of the feeding cycle and the circadian cycle that involve hormones that exhibit circadian rhythmicity such as leptin, ghrelin, cortisol and melatonin. Such interaction balances hormonal levels to normalize physiological processes. In a situation where an individual experiences disrupted circadian rhythm (for example by not sleeping sufficiently through the night), such individual suffers an accompanying decrease in leptin levels. According to an embodiment of the claimed invention, the normalizing of the circadian activity is correlated with relatively higher levels of leptin. Accordingly, embodiments of the claimed invention can be used in as, or as part of, weight loss therapy.

According to an embodiment of the claimed invention, the apparatus and method prevent circadian-disrupting spectrums from reaching photoreceptors by filtering out circadian-disrupting wavelengths emitted from electronic devices, thereby preventing those spectrums from reaching such photoreceptors which may act in altering biological rhythms.

Photoreceptors are contained in human eyes as well as human skin. Therefore, embodiments of the present invention can be utilized so that circadian disrupting light is shielded from photoreceptors in the skin while continuing the transmission of proper circadian promoting light. Embodiments of the current invention herein are configured to maximally shield, among others photoreceptors, rods and cones and the photosensitive ganglion cells.

According to an embodiment of the present invention, the optical device employed in the method may cover or shield the body from light emitting sources by protecting the photoreceptors in the subject's skin. The skin's pigmentation is the result of melanin produced by the melanocyte cells. A gene expression profile of melanocytes has revealed that such skin cells also express rhodopsin, a light sensitive chemical and photoreceptor predominately found in the retina. By limiting light to the photoreceptors in the skin, circadian disruptive signals are thwarted from communicating to the central and peripheral clocks regulating the light-dark cycle.

According to an embodiment of the claimed invention, the optical devise employed in the method can be fashioned into objects including eyewear, sleep masks, light filters or covers fashioned over light emitting or transmitting objects including computers, tablets, cell phones, electronic devices, televisions, windows, and other device etc.

According to an embodiment of the claimed invention, the optical device is a lens.

According to an embodiment of the present invention, the optical devise may be incorporated within a variety of eyewear including, but not limited to: eye glasses including prescription and non-prescription eyewear, sleep masks, fit-over glasses that can be placed on top of prescription and other glasses, swimming goggles, foldable eyewear, photochromic, color changing eyewear, and may have insertable, removable or clip on darkening shades or any other type of eye wear made out of any transparent or semi-transparent material including glass, gels or plastic.

According to an embodiment of the apparatus, the optical device is configured to be a red optical filter of transparent or semi-transparent material (e.g. glass or plastic). Such material can be molded in a way to shield the photoreceptors including photosensitive ganglion cells and rods & cones in the eye from light from a specified wavelength while allowing only the varying colors of fire light (i.e. transmit or are colored at least one of the following spectrums: infrared, near infrared, red, yellow and orange) to penetrate in the objects made from the material.

According to an embodiment of the current invention, a filter (201) group of the optical device may be comprising of a yellow filter and a magenta filter to be used such that only red light is able to pass through while blocking violet, blue and green light.

According to an embodiment of the present invention, the percent of transparency may be altered and made darker or lighter to suit the functionality and safety needs of the user. Lighter transparency provides more functionality; darker transparency provides more of a therapeutic effect.

According to embodiments of the present invention, the optical device may be configured as bifocal eyewear. Without any limitation, embodiments of bifocal eyewear comprising of the present invention may be in the style of round top, flat top, curved top or ribbon segments, split top, and/or fused.

According to embodiments of the present invention the optical device may be configured as prescription eyewear.

According to embodiments of the present invention, the optical device may be tinted. In one embodiment, the optical device is tinted red. In another embodiment, the optical device is tinted pink and/or rose-colored.

According to embodiments of the present invention, the optical device may be plastic screens for placing over light-emitting electronic devices, windows, and windshields. One of ordinary skill in the art would appreciate that there are numerous form factors for light filtering devices that could be utilized with embodiments of the present invention, and embodiments of the present invention are contemplated for use with any appropriate form factor.

According to an embodiment of the present invention, the optical device may be a pair of glasses that can wrap around the eyes as well as the temple. This has the effect of blocking any incoming light from all directions.

While multiple embodiments are disclosed, still other embodiments of the present invention will become apparent to those skilled in the art from this detailed description. The invention is capable of myriad modifications in various obvious aspects, all without departing from the spirit and scope of the present invention. Accordingly, the drawings and descriptions are to be regarded as illustrative in nature and not restrictive.

According to an embodiment of the present invention, the method provides darkness therapy by blocking at least 50%, at least 60, at least 70%, at least 80, and at least 90% of the light transmitted to an individual's eyes and/or skin.

According to an embodiment of the present invention, the method additionally blocks blue light (450 nm-495 nm) as well as green light (495-570 nm).

According to an embodiment of the present invention, the method additionally blocks violet light (380 nm-495 nm) as well as blue and green light (495-570 nm).

According to an embodiment of the present invention, the method additionally blocks UV light (below 380 nm) and blocks violet, blue, green and yellow light (380-590 nm).

According to an embodiment of the present invention, the method additionally blocks UV light (below 380 nm) and blocks violet, blue, green, yellow and orange light (380-620).

Certain embodiments of the present invention may also be configured to block or allow UV light, depending on application and desired effect. The effect of this is that manifestations of this invention may significantly regulate circadian effect in promoting sleep and night time circadian rhythms than prior art, as determined by the spectral sensitivity of the intrinsically photosensitive ganglion cells that contain melanopsin.

According to an embodiment of the claimed invention, the method can more comprehensively and healthfully alter circadian and biological rhythms of a subject, with fewer side effects than prior art by providing light therapy that transmits long-wavelength light (defined as transmitting at least one of the following spectrums: red, near infrared, infrared) spectrums and blocks short or short and middle range wavelengths of light (defined as light transmitting at least one of the following spectrums: green, blue, and violet) that most adversely affect human health if exposed at night or during other desynchronous light periods such as during the day for shiftworkers that work during the night and sleep during the day.

According to an embodiment of the claimed invention, the method is configured to permit the human body to tune with natural light patterns by transmuting the spectrums of light that have the most circadian stimulating effect into shades of firelight which has the least circadian stimulating response. Firelight is defined as the resulting light after blocking exposure to the wavelengths of light contained in green spectrums (495-570 nm) and below.

Red is the longest spectrum of light visible to the human eye, and thusly it is the last spectrum of light to be seen as the sun sets. This red sunset signal may ready the body to begin shifting into a night time circadian rhythm and Dim Light Melatonin Onset (DLMO). According to an embodiment of the claimed invention, the method comprises of the transmission of long-wavelength light in the absence of short wavelengths of light, thereby, stimulating a “virtual sunset” signal to the circadian receptors to begin DLMO and to begin producing night time circadian rhythms and associated hormones, including melatonin, leptin, ghrelin and at least one glucocorticoid (including cortisol).

The red and near infra red light that can be transmitted via embodiments of the present method have been shown to be most beneficial to lowering inflammation, promoting cell health and growth, healing from tissue damage and regeneration. Embodiments of the present invention may also provide immune system modulating spectrums to the retina and other parts of the body that can include red light therapy, near infrared light therapy, or infrared light therapy by concentrating those spectrums of light and significantly blocking other wavelengths from reaching the eye.

According to an embodiment of the claimed invention, the method is used in coordination with light therapy during the day (or as appropriate for nighttime shift workers to coincide with peak activity periods. While the subject receives light therapy, the method blocks blue/green light and transmits at/or near infrared light. This allows the subject to heal from the ailments targeted by the light therapy while simultaneously promoting healthy circadian rhythms. By substantially blocking a highly stimulatory wavelength of light found in short and moderate spectrums, while simultaneously providing long wavelength light therapy (defined as providing at least one of the following spectrums: red, near infrared, infrared light) embodiments of the present invention may provide an effect approaching virtual darkness and mimic the fire-like spectrum of light that diurnal mammals like humans are best adapted to see at night.

Intrinsically photosensitive ganglion cells (IPGC), which contain melanopsin are circadian photopigments important to setting circadian rhythms. Exposure of melanopsin containing IPGC to green light may stimulate mammals to delay producing night time circadian rhythms and stimulate wakefulness.

Prior technology does not sufficiently address the role of IPGC in setting a circadian rhythm. The complete blocking of light, not just blocking of light to photoreceptors, has been argued to set the pace of the circadian clock, located in the supra-chiasmatic nuclei (SCN) in the brains hypothalamus. Embodiments of the present invention seek to provide for blocking the full range of night-time deleterious light spectrums and providing an effect approaching virtual darkness to circadian receptors by blocking the most deleterious wavelengths for night time exposure while simultaneously transmitting wavelengths with the least deleterious effect for night time exposure or, in the alternative, provide optional degrees of light for functional purposes. As measured on a logarithmic scale of signaling for melanopsin, which is arguably the primary circadian time setter, the circadian efficacy of this invention increases by orders of magnitude as compared with existing prior art via its ability to block spectrums sensitive to IPGC which prior art transmit to the photoreceptors of the eye.

According to an embodiment of the claimed invention, the method can be used by subjects suffering from diabetes to promote sleep, melatonin production and thus increased insulin sensitivity. Sleep efficiency or effectiveness is substantially reduced in people with diabetes and metabolic syndrome, as research has shown that diabetics have significantly less melatonin production than healthy people, and may be prone to difficulties in sleep. People with diabetes also produce less melatonin during the night than healthy people.

Sleep loss, light at night and circadian rhythm disruption is also associated with increased risk for various cancers, including hormone specific cancers such as breast and prostate cancer. Accordingly, embodiments of the current invention can be used to block disruptive circadian stimuli to promote healthful rest.

According to an embodiment of the claimed invention, the method is used to provide therapeutic treatment with long wavelength to the skin or eye. A study has found that photobiomodulation with near infrared light “is protective against bright-light-induced retinal degeneration, even when near infrared light treatment is applied after exposure to light. This protective effect involves a reduction of cell death and inflammation. Photobiomodulation has the potential to become an important treatment modality for the prevention or treatment of light-induced stress in the retina. More generally, it could be beneficial in the prevention and treatment of retinal conditions involving inflammatory mechanisms.”

According to an embodiment of the present invention, the method may also be used as part of dark therapy, also called scototherapy or light restriction, treatment while allowing its users to continue to see and operate functionally for various conditions such as headaches or bipolar mania. Scototherapy is helpful and beneficial to a variety of ailments, including but not limited to, affective disorders, bipolar disorder and cancer. Scototherapy may also manipulate biological and circadian rhythms by altering rhythms and production of hormones and neurotransmitters. Embodiments of present invention may also have the means to provide virtual darkness. The manipulation of light enabled by this invention and along with the sleep/wake therapies may provide a treatment for psychological conditions such as depression and other affective condition. Such treatment may also include the use of drug medication. Scototherapy and biological rhythm optimization can be a helpful tool for multiple affective disorders, not just bipolar.

According to an embodiment of the present invention as a scototherapy, an individual is instructed to gain bright light exposure in the morning. Light exposure can be either through sun exposure or a light lamp providing 10,000 lux of full spectrum light (scotopic light measured at 10 inches). Following light exposure, the individual is directed to eat a breakfast comprising of protein. The individual is able to eat any other subsequent meals or snacks, but must refrain from eating 4-6 hours before bedtime. Following sunset, the individual is instructed to wear appropriate clothing to shield the skin from artificial light. For 2-4 hours prior to bed time, the individual is then required to cover his eyes with an optical device comprised of red-colored lenses block at least 90% or more light at wavelengths in the range of 495-570 nm (+/−15 nm) and transmit light at red wavelengths (620-750 nm).

According to an embodiment of the present invention, the method may assist with health issues associated with circadian disruption and from light at night, including alterations in natural feeding rhythms. For example, circadian disruption may cause night time emotional eating and food cravings including carbohydrate cravings in subjects that are exposed to light at discordant circadian light intervals (e.g. for subjects operating on nontraditional schedules like shift workers). Studies have shown that mice exposed to extra light during periods where they should be sleeping put on nearly 50 percent more weight than mice fed the same diet who lived on a natural light and rhythm. Sleeplessness and stress, for instance, have been linked to disturbances in the effects of leptin and ghrelin, the hormones that tells the brain whether more food is required or if the body has had enough to eat. Blue light spectrum's transmission to the retinal photoreceptors stimulates the body's hunger hormones, namely increasing leptin and lowering ghrelin to encourage feeding and hunger signaling. In a preferred embodiment, the light filtering device blocks retinal absorption of spectrums of light with the means of relatively lowering leptin and relatively increasing ghrelin. In the preferred embodiment, this is accomplished by transmitting red light and/or near infrared light (which has relatively least disruptive effect on leptin and ghrelin and blocking at least one of the following spectrums: violet, blue, green, yellow or orange light. In alternate preferred embodiment, the light filtering device regulates and normalizes the hormones involved in hunger, feeding, weight management and other related functions. As specific wavelengths of light have been shown to modulate hormones such as leptin and ghrelin, both of which are associated with modulation of hunger signaling and weight homeostasis; the ubiquity of light at night may contribute to the widespread rise of obesity and overweight, this invention may be used to mitigate this negative effect of light at night to promote healthier eating patterns and conditions. For example, by regulating and lowering a subject's hunger signaling chemicals, the method may be used to treat leptin resistance, overweight and obesity, as red light has the most beneficial effects on nocturnal biological and circadian hormonal normalization as dim red light approaches biologically similar effects on hunger associated hormones such as leptin and ghrelin to approach the effect of darkness on leptin and ghrelin, relative to the wavelengths the invention blocks such as blue and green light. This promotes the means to facilitate the normalization of leptin and ghrelin at night which is important to leptin and insulin sensitivity which is critical to various conditions including metabolic syndrome, diabetes, cardiovascular disease, regulating weight homeostasis and many other issues and conditions.

According to an embodiment of the present invention, the method may be used to regulate hormones associated with hunger. In a preferred embodiment, the method may lower ghrelin. Ghrelin is a gastric hormone associated with hunger, that, when rising, may stimulate hunger. In an alternate preferred embodiment of the present invention, the light filtering device may increase leptin. Leptin is a hormone that, when rising, may lower hunger. In further preferred embodiment the method may affect the levels of both ghrelin and leptin simultaneously or independently. As an illustrative example, by increasing leptin and lowering ghrelin a preferred embodiment of this invention would have the means to simultaneously increase satiety hormone patterns (leptin) while lowering hunger hormone patterns (ghrelin), which would in turn increase the desire for sleep, as sleeping and eating are generally mutual exclusive. Light is a potent zeitgeber or biological rhythm timegiver to set circadian and feeding rhythms. Human ancestors did not have light at night and slept at night thus did not eat well into the night. By increasing the feeding window via light at night this increases signaling for the body to eat and allows for more energy to be consumed. Also the alteration of leptin levels by light may contribute to leptin resistance which is associated with obesity through disruptions in weight and caloric balance homeostasis. The widespread use of light at night and electrification of society may be contributing to the promotion of obesity and this invention and the embodiments therein may be a tool to mitigate some of the deleterious effects of light at night. This invention may thus be used in part to help restore leptin sensitivity. Similarly it may used during other times virtual darkness is beneficial such as during the day for people who live and work during night periods such as shift workers. The invention may also be at any time to facilitate virtual darkness when experiencing a light sensitive headache. Embodiments of the present invention may help users address issues that include, but are not limited to, night time eating syndrome, overeating, overweight, emotional eating, obesity, binge eating, leptin resistance, insulin resistance, diabetes, metabolic syndrome, cancer, psychological conditions, headaches (including light sensitive headaches and migraines), enriching breast milk with melatonin, lowering glucocorticoids in breast milk and more.

According to an embodiment of the present invention, the method may be used to lower the palatability of food. In general, foods devoid of color have less perceived palatability, and foods of different colors may provide different perceptions of taste even though they may not have differences aside from the color. The color of food has been shown to affect sweetness perception. Foods colored red appear sweeter to the eye, as studies have shown that drinks of identical sugar content are perceived to be even sweeter when colored red. The effect of color is believed to be due to cognitive expectations. In a preferred embodiment of the present invention, certain spectrums of light could be blocked to decrease the perceived palatability of food. One of ordinary skill in the art would appreciate that various light spectrums can be blocked while transmitting different wavelengths of light including those found in the red spectrum in order to decrease the palatability of high energy food or increasing the palatability of lower energy food to assist in weight management. Embodiments of the present invention are may be configured to block any spectrum of light that would contribute to decreasing food palatability.

According to an embodiment of the present invention, the method may be used to modulate the circadian rhythms of breast feeding mothers. In a preferred embodiment, utilizing various embodiments of this invention, selective blocking of lower wavelengths and transmission of longer wavelengths of light spectrums for at least two to four hours before feeding pumping breastmilk may increase melatonin production and may increase melatonin in the breast milk of breast feeding mothers. The mother's melatonin and glucocorticoids like cortisol can migrate from general circulation and move into mother's milk. By pumping or feeding mother's milk after utilizing this method, a breast feeding mother can transmit melatonin in the milk to help communicate time of day information and promote proper synchronization of the child's circadian rhythm with the effect of promoting sleep. Alternatively, this invention may be used to lower the glucocorticoid content in mothers milk. In a preferred embodiment of this invention melatonin content may be increased and cortisol content may be decreased in a breast feeding mother.

According to an embodiment of the present invention, the method may be used by pregnant women that are exposed to light at night. In the preferred embodiment, using the light filtering device helps to instill healthy circadian rhythms in their offspring during prenatal development.

The human body has a diurnal rhythm of core body temperature regulation. The circadian rhythm of core body temperature is a well-documented physiological phenomenon. It is observed that core body temperature is highest at around 6 pm with a high of 37 degrees Celsius and a low of about 36.2 degrees Celsius. The effects of light on the behavior of core temperature and melatonin vary depending on its wavelength. Light with long wavelengths, specifically the red color spectrums had little influence on the human temperature rhythms. However, the green spectrum and blue light had a great influence. In one study blue light was able to inhibit the fall of night time body temperature by 60% and green light has been shown to inhibit the fall by 80%.

According to an embodiment of the present invention, the method may assist in regulating core body temperature. In the preferred embodiment, the method may be used to regulate core body temperature by substantially inhibiting blue and green light. In an alternate preferred embodiment, the method may be used to provide virtual darkness therapy, wherein the light filtering device may modulate core body temperature through the significant filtration of wavelengths found in green light and below. By significantly blocking out of the wavelengths including blue and the green spectrums from reaching retinal photoreceptors and significantly transmitting long wavelength light (at least one of the following spectrums: orange, red and/or near infrared light spectrums), this has effect of facilitating normalizations in nighttime core body temperature to lower night time core body temperature of a subject.

According to an embodiment of the present invention, the method may also be used during daylight hours. In an alternate embodiment, the method may be used to help the user adjust to jet lag. In another embodiment, the method may be used by shift workers to adjust to sleeping during the day by blocking alerting wavelengths such as those found in blue and green spectrums found in natural sunlight and ultraviolet light to assist in adjusting biological and sleep rhythms to sleeping during the day by blocking naturals sunlight and ultraviolet light and thus provide darkness therapy.

According to an embodiment of the present invention, the optical device may need to be worn, applied or activated (continuously for several hours, often at least 2-4 hours for some of the effects can take place, other effects may take shorter time periods.

According to an embodiment of the present invention, the method may be used in healthy people without medical condition for health promoting or prevention purposes.

According to an embodiment of the present invention, the method may improve sleep quality and circadian rhythms, which promotes healthful lifestyles, as sleep is restorative and promotes immune system health. In a preferred embodiment of the present invention, method may be a choice that promotes healthier lifestyles for people facing or seeking to prevent conditions that include, but are not limited to insomnia, obesity, leptin resistance, insulin resistance, diabetes, stroke, cancer, cardiovascular disease, retinal inflammation, macular degeneration, cognitive impairment, depression, bi-polar disorder, seasonal affective disorder, attention deficit hyperactive disorder, jet lag, migraines, HIV, anxiety, panic disorder, binge eating disorders, night time eating syndrome, Alzheimer's, infection, schizophrenia, suicidal behavior, gastrointestinal disorders, liver disease, autoimmune conditions, alcohol addiction, drug addiction, Parkinson's disease, hepatitis, and increasing low melatonin content in breast milk to promote sleep and health in breast fed children, as well as many more issues and conditions that would be obvious to one of skill in the art.

According to an embodiment of the present invention, the method may be used during the day, or at other times with ample ambient light, to alter various chronotypes to allow people with increased eveningness commonly known as “night owls” to better adapt to more morningness commonly known as becoming more “early birds”. In addition children with ADHD have increased rates of eveningness and staying up later and exposing themselves to short and moderate wavelength light may lower their sleep quality and quantity which is very important in children with ADHD because at night during the dark while they sleep dopamine receptors become most numerous. Thus, sleep deprivation including chronic sleep deprivation reduces dopamine D2/D3 receptor availability in both healthy people and people with ADHD. With this invention helping to instill less eveningness and better sleep hygiene it may help in part with the management of ADHD symptoms in children and adults.

According to an embodiment of the present invention, the method may be utilized during the night or other times for optimizing healthy circadian rhythm.

According to an embodiment of the present invention, the method may be used to facilitate virtual darkness at any time when the wearer can benefit, including for headaches or to provide long wavelength therapy to the eye.

According to an embodiment of the present invention, the method may be used to facilitate virtual darkness that may be therapeutic for myriad psychological and biological conditions including bipolar disorders and bipolar mania.

The biological rhythm system through its evolution was designed to deal with differing needs of the body depending not only on time of day as in circadian rhythms, but also the time of year with patterns of different hormone levels in the seasons such variations in testosterone, thyroid and more. The human body has seasonal variations in hormonal production called circannual rhythms. This hormonal regulation may be signaled by the amount of light available and the amount of melatonin produced. By creating a circadian virtual darkness effect, embodiments of the present invention may be utilized to not only modulate daily circadian rhythms but also seasonal rhythms by utilizing the invention for longer periods during the winter when light exposure is naturally shorter. A subject can mimic seasonal circannualrhythms by applying an embodiment of the present invention once the sun sets, with less daily utilization in the summer months and more utilization in the winter months.

According to an embodiment of the present invention, the method is designed to minimize the deleterious effects of light at night or light during the day for people utilizing alternate schedules such as shift workers. In this manner, the method filters natural (sunlight with UV spectrums) as well as filtering spectrums of artificial lighting and transmuting them via a red optical filter into spectrums of light naturally contained in fire.

According to an embodiment of the present invention, the method is designed to reset master clock before/during/after jet lag. The individual is instructed to wear the optical device in the days or weeks prior to a flight and further instructed to wear the optical device.

1. A method for providing darkness therapy for altering biological rhythms comprising:
covering an individual's eyes with an optical device, configured to block at least 90% or more light at wavelengths in the range of 495-570 nm and transmit light in at least one of orange (590-620 nm), red (620-750 nm), and near infrared light range spectrum (750 nm or greater), wherein said eyes are covered by said optical device for 2-4 hours before bedtime to normalize said individual's circadian rhythm,
wherein said optical devise comprises of red-colored lenses.

2. The method of claim 1, further configured to block at least 90% or more light at wavelengths in the range of 450 nm-495 nm.

3. The method of claim 1, further configured to block at least 90% or more light at wavelengths in the range of 380 nm-495 nm.

4. The method of claim 3, further configured to block at least 90% or more light at wavelengths below 380 nm.

5. The method of claim 1, to increase the level of at least one or more of melatonin.

6. The method of claim 1, to increase the level of at least one or more of leptin.

7. The method of claim 1, to lower at least one corticosteroid such as cortisol.

8. The method of claim 1, to lower the hormone ghrelin.

9. The method of claim 1, to assist with at least one of weight management, sleep management, relaxation or stress management.

10. The method of claim 1, to treat at least one eating disorder including binge eating disorder and nighttime binge eating disorder.

11. The method of claim 1, for enriching melatonin in breast milk in a lactating woman wherein said optical device is work for at least two to four hours and longer at night.

12. The method of claim 1, for use in treating jet lag wherein the optical device is worn an hour earlier every week or every day to prepare body before flight,
wherein the individual is further instructed to condition said body by using the device for at least 2-4 hours prior to said person's normal bedtime rest,
wherein the individual is further instructed to condition said body to adjust to said jet lag by using the device for at least 2-4 hours daily before bedtime until regular sleep pattern resumes.

13. The method of claim 1, for use in add-on treatment in a class of affective disorders, including bipolar disorder, anxiety, and depression.

14. An optical device for providing darkness therapy for altering biological rhythms comprising:
a filter, configured to block at least 90% or more light at wavelengths in the range of 495-570 nm and transmit light in at least one of yellow (570-590 nm), orange (590-620 nm), red (620-750 nm), and near infrared light range spectrum (750 nm or greater),
wherein said filter is a red-colored lens.

15. The apparatus of claim 14, further configured to block at least 90% or more light at wavelengths in the range of 450 nm-495 nm.

16. The apparatus of claim 14, further configured to block at least 90% or more light at wavelengths in the range of 380 nm-495 nm.

17. The apparatus of claim 14, further configured to block at least 90% or more light at wavelengths below 380 nm.

18. The apparatus of claim 14, wherein said device is embodied in a pair of glasses.

19. The apparatus of claim 18, wherein said glasses wrap around an individual's temple.
